(19) United States
(12) Patent Application Publication
(10) Pub. No.: US 2022/0323569 A1
(43) Pub. Date: Oct. 13, 2022

(54) PLANT-PRODUCED VLPS AND RIC VACCINES

(71) Applicant: Qiang Chen, Chandler, AZ (US)

(72) Inventor: Qiang Chen, Chandler, AZ (US)

(21) Appl. No.: 17/708,797

(22) Filed: Mar. 30, 2022

Related U.S. Application Data
(60) Provisional application No. 63/168,135, filed on Mar. 30, 2021.

(51) Int. Cl. A61K 39/145; C12N 15/82; C12N 7/00; A61P 31/14

(52) U.S. Cl. CPC A61K 39/145; C12N 15/8203; C12N 15/8258; C12N 7/00; A61P 31/14; A61K 2039/517

(57) ABSTRACT

Severe Acute Respiratory Syndrome Coronavirus 2 antigen virus-like particles (VLPs) and recombinant immune complexes (RICs) are described, along with methods of making said VLPs and RICs in plants and using said VLPs and RICs to induce an immune response in a subject.

### CROSS-REFERENCE TO RELATED APPLICATIONS

This patent application claims the benefit of priority of U.S. Provisional Patent Application No. 63/168,135 filed on Mar. 30, 2021 which is incorporated herein by reference in its entirety.

### SEQUENCE LISTING

This application is being filed electronically via EFS-Web and includes an electronically submitted Sequence Listing in .txt format. The .txt file contains a sequence listing entitled “112624_01333_ST25.txt” created on Mar. 30, 2022 and is 92,956 bytes in size. The Sequence Listing contained in this .txt file is part of the specification and is hereby incorporated by reference herein in its entirety.

### BACKGROUND

Coronaviruses (CoV) constitute a large family of positive-stranded, enveloped RNA viruses that infect a broad range of mammalian and avian species. The viruses cause primarily respiratory and enteric diseases. In the last two decades three new zoonotic CoVs have emerged to infect humans. The most recent emergence of SARS-CoV-2 that continues to spread globally raises many scientific and public health questions and challenges. Development of effective vaccines and antiviral therapeutics and rapidly deployment of both is still a pressing need. Previous work with the other two recent emergent pathogenic human CoVs, severe acute respiratory syndrome (SARS-CoV) and Middle East respiratory syndrome (MERS-CoV), provides insight and platforms that can help expedite the process of vaccine development, but safe, effective, inexpensive vaccines are still needed. There are currently seven CoVs that infect humans, HCoVs 0C43, 229E, NL63 and HKU1, that cause seasonal upper respiratory infections, in addition to the three more pathogenic viruses. The human viruses are thought to have emerged from zoonotic hosts to infect humans. Viral genomic analyses indicate that the human viruses are related to bat CoVs. A large number of novel CoVs have been identified in bat populations since identification of SARS-CoV and the expectation is that we will continue to have spillover of these viruses to humans. This reinforces the need for development of vaccines against emergent CoVs.

### SUMMARY OF THE INVENTION

In a first aspect, provided herein is a geminiviral vector comprising a polynucleotide encoding a Severe Acute Respiratory Syndrome Coronavirus 2 (SARS-CoV-2) antigen; and a polynucleotide encoding Hepatitis B core antigen (HBc). In some embodiments, the geminiviral vector is based on bean yellow dwarf virus (BeYDV) genome. In some embodiments, the polynucleotide encoding a SARS-CoV-2 antigen is codon optimized for Nicotiana benthamiana. In some embodiments, the SARS-CoV-2 antigen is selected from the group consisting of SARS-Cov-2 spike (S) protein (SEQ ID NO:1), mutant SARS-CoV-2 S (SEQ ID NO: 18), SARS-CoV-2 receptor binding domain (RBD) (SEQ ID NO:6), SARS-CoV-2 receptor binding domain mutants (Table 1 and SEQ ID NO: 22-41), SARS-CoV-2 membrane (M) protein (SEQ ID NO:7), SARS-CoV-2 envelope (E) protein (SEQ ID NO:8), SARS-CoV-2 nucleocapsid (N) protein (SEQ ID NO:9), sequences at least 90% identical thereto, and combinations thereof.

In a second aspect, provided herein is a method for producing a SARS-CoV-2 VLP comprising expressing in a plant a vector described herein; and purifying the SARS-CoV-2 VLP from the plant. In some embodiments, the plant is Nicotiana benthamiana. In some embodiments, the vector is transfected into a leaf of the plant. In some embodiments, the method comprises expressing in the plant at least two vectors each encoding a different SARS-CoV-2 antigen. In some embodiments, the method comprises expressing in the plant at least three vectors each encoding a different SARS-CoV-2 antigen. In some embodiments, the VLP is purified using chromatography. In some embodiments, the plant is transfected with the vector and the VLP is purified at least 3 days after transfection.

In a third aspect, provided herein is a SARS-CoV-2 VLP. In some embodiments, the VLP comprises a SARS-CoV-2 antigen selected from the group consisting of SARS-Cov-2 spike (S) protein (SEQ ID NO:1), mutant SARS-CoV-2 S (SEQ ID NO: 18), SARS-CoV-2 receptor binding domain (RBD) (SEQ ID NO:6), SARS-CoV-2 receptor binding domain mutants (Table 1 and SEQ ID NO: 22-41), SARS-CoV-2 membrane (M) protein (SEQ ID NO:7), SARS-CoV-2 envelope (E) protein (SEQ ID NO:8), SARS-CoV-2 nucleocapsid (N) protein (SEQ ID NO:9), sequences at least 90% identical thereto, and combinations thereof.

In a fourth aspect, provided herein is a vaccine composition comprising a SARS-CoV-2 VLP described herein and a pharmaceutically acceptable carrier. In some embodiments, the vaccine comprises an adjuvant.

In a fifth aspect, provided herein is a method of inducing an immune response in a subject against a SARS-CoV-2 antigen comprising administering an effective amount of a vaccine composition described herein to the subject. In some embodiments, the SARS-CoV-2 antigen is selected from the group consisting of SARS-Cov-2 spike (S) protein (SEQ ID NO:1), mutant SARS-CoV-2 S (SEQ ID NO: 18), SARS-CoV-2 receptor binding domain (RBD) (SEQ ID NO:6), SARS-CoV-2 receptor binding domain mutants (Table 1 and SEQ ID NO: 22-41), SARS-CoV-2 membrane (M) protein (SEQ ID NO:7), SARS-CoV-2 envelope (E) protein (SEQ ID NO:8), SARS-CoV-2 nucleocapsid (N) protein (SEQ ID NO:9), sequences at least 90% identical thereto, and combinations thereof. In some embodiments, the SARS-CoV-2 antigen is SARS-CoV-2 S protein (SEQ ID NO:1) or a sequence 90% identical thereto. In some embodiments, the subject is a human.

In a sixth aspect, provided herein is a vector comprising a first polynucleotide encoding a SARS-CoV-2 antigen; a second polynucleotide encoding an immunoglobulin heavy chain; and a third polynucleotide encoding an epitope tag that binds to the immunoglobulin heavy chain, wherein the first, second, and third polynucleotides are arranged in the vector such that, when expressed from the vector, the SARS-CoV-2 antigen is between the C-terminus of the immunoglobulin heavy chain and the N-terminus of the epitope tag to form a recombinant immune complex (RIC). In some aspects, the vector is a geminiviral vector. In some embodiments, the geminiviral vector is based on bean yellow dwarf virus (BeYDV) genome. In some embodiments, the polynucleotide encoding a SARS-CoV-2 antigen is codon optimized for Nicotiana benthamiana. In some embodiments, the SARS-CoV-2 antigen is selected from the group consisting of SARS-Cov-2 spike (S) protein (SEQ ID NO:1), mutant SARS-CoV-2 S (SEQ ID NO: 18), SARS-CoV-2 receptor binding domain (RBD) (SEQ ID NO:6), SARS-CoV-2 receptor binding domain mutants (Table 1 and SEQ ID NO: 22-41), SARS-CoV-2 membrane (M) protein (SEQ ID NO:7), SARS-CoV-2 envelope (E) protein (SEQ ID NO:8), SARS-CoV-2 nucleocapsid (N) protein (SEQ ID NO:9), sequences at least 90% identical thereto, and combinations thereof.

In a seventh aspect, provided herein is a method for producing a SARS-CoV-2 recombinant immune complex (RIC) comprising expressing in a plant a vector described herein; and purifying the SARS-CoV-2 RIC from the plant. In some embodiments, the plant is Nicotiana benthamiana. In some embodiments, the vector is transfected into a leaf of the plant.

In an eight aspect, provided herein is a SARS-CoV-2 RIC produced by the methods described herein. In some embodiments, the RIC includes the SARS-CoV-2 antigen positioned between the C-terminus of the immunoglobulin heavy chain and the N-terminus of the epitope tag. In some embodiments, the SARS-CoV-2 antigen is selected from the group consisting of SARS-Cov-2 spike (S) protein (SEQ ID NO:1), mutant SARS-CoV-2 S (SEQ ID NO: 18), SARS-CoV-2 receptor binding domain (RBD) (SEQ ID NO:6), SARS-CoV-2 receptor binding domain mutants (Table 1 and SEQ ID NO: 22-41) SARS-CoV-2 membrane (M) protein (SEQ ID NO:7), SARS-CoV-2 envelope (E) protein (SEQ ID NO:8), SARS-CoV-2 nucleocapsid (N) protein (SEQ ID NO:9), sequences at least 90% identical thereto, and combinations thereof.

In a ninth aspect, provided herein is a vaccine composition comprising a SARS-CoV-2 RIC described herein and a pharmaceutically acceptable carrier. In some embodiments the vaccine comprises an adjuvant.

In a tenth aspect, provided herein is a method of inducing an immune response in a subject against a SARS-CoV-2 antigen comprising administering an effective amount of a vaccine composition described herein to the subject. In some embodiments, the SARS-CoV-2 antigen is selected from the group consisting of SARS-Cov-2 spike (S) protein (SEQ ID NO:1), mutant SARS-CoV-2 S (SEQ ID NO: 18), SARS-CoV-2 receptor binding domain (RBD) (SEQ ID NO:6), SARS-CoV-2 receptor binding domain mutants (Table 1 and SEQ ID NO: 22-41), SARS-CoV-2 membrane (M) protein (SEQ ID NO:7), SARS-CoV-2 envelope (E) protein (SEQ ID NO:8), SARS-CoV-2 nucleocapsid (N) protein (SEQ ID NO:9), sequences at least 90% identical thereto, and combinations thereof. In some embodiments, the SARS-CoV-2 antigen is SARS-CoV-2 S protein (SEQ ID NO:1) or a sequence at least 90% identical thereto. In some embodiments, the subject is a human.

### BRIEF DESCRIPTION OF DRAWINGS

FIG. 1 shows the DNA and amino acid sequences of HBcAg-SARS-CoV-2-RBD, which comprises the HBc gene with the coding sequence of the receptor-binding domain (RBD) of the S protein inserted into the major insertion region (MIR).

FIG. 2 shows the DNA and amino acid sequences of NVCP-SARS-CoV-2-RBD, which comprises the RBD of the S protein inserted into the Norwalk virus capsid protein (NVCP).

FIG. 3 shows a western blot analysis of NVCP-SARS-CoV-2-RBD produced in plants. The membrane was incubated with antibodies against NVCP. The size of NVCP-SARS-CoV-2 RBD is predicted to be ˜80 kDa. Noninfiltrated plant extract=negative control, infiltrated plant extract=NVCP-RBD crude extract, before gradient=concentrated NVCP-RBD sample.

FIG. 4 shows the percent of original body weight of mice following vaccination and challenge with a mouse-adapted SARS-CoV-2 virus. Mice were vaccinated with a prime and two boots of the indicated vaccines and body weight was measured for 10 days post-challenge.

FIG. 5 shows clinical scores for the mice tested in FIG. 8.

FIG. 6 shows dilutions of the serum of mice treated with a prime of a plant-derived VLP and two boosts of a replication-competent vaccine vector (i.e., group B).

FIG. 7 shows the SARS-CoV-2 neutralizing antibody activity detected in the serum of vaccinated mice at five time points: before prime, one month after prime, one month after the 1st boost, three months after the 1st boost, and two weeks after the 2nd boost.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### DETAILED DESCRIPTION OF THE INVENTION

The present disclosure describes Severe Acute Respiratory Syndrome Coronovirus 2 (SARS-CoV-2) vaccine compositions, as well as methods for making and using the same. Severe acute respiratory syndrome coronavirus 2 (SARS-CoV-2) is the third highly pathogenic human CoV to emerge in the past two decades. The virus causes COVID-19, a severe respiratory disease with an estimated mortality of 2-3% that rapidly spread across China beginning in late 2019. The virus also spread globally and was declared a pandemic in 2020. Like other CoVs, the spike (S) protein is assumed to be the major target for neutralizing antibodies. SARS-CoV-2 S protein binds to the receptor, angiotensin-converting enzyme 2(ACE2), through its receptor binding domain (RBD). The RBDs for other CoVs are immunogenic and a major neutralizing determinant. There are significant concerns that the virus will be embedded in the viral respiratory disease landscape that will be encountered seasonally. Thus, development of a safe, effective, inexpensive and widely distributed vaccine against the virus is a significant priority. The long-term goal of this project is to develop a safe, efficacious vaccine(s) against SARS-CoV-2. Standard molecular biology, biochemical approaches, vaccination and immunogenicity assessment will be used to generate virus-like-particles (VLPS) in mammalian cells. The goal of this work is to optimally produce plant derived VLPs and RICs vaccines and evaluate immune responses elicited in mice vaccinated with the VLPS.

SARS-CoV-2 includes membrane (M), spike (S), envelope (E), and nucleocapsid (N) structural proteins. The M, S, and E proteins provide the structure of the exterior viral envelope. The S protein is a glycoprotein that mediates receptor binding and fusion during entry into a host cell. The S protein of SARS-CoV-2 has the sequence of SEQ ID NO:1. The receptor binding domain (RBD, SEQ ID NO:6) is amino acids 318-510 of SEQ ID NO:1. SEQ ID NO: 18 is a mutated S protein with stabilizing mutations. SEQ ID NO: 22-41 are RBD sequences with mutations found in SARS-CoV-2 variants (See Table 1 below). The M protein of SARS-CoV-2 has the sequence of SEQ ID NO:7. The E protein of SARS-CoV-2 has the sequence of SEQ ID NO:8. The N protein is an internal structural component that encapsulates the SARS-CoV-2 viral genome. The N protein of SARS-CoV-2 has the sequence of SEQ ID NO:9.

TABLE 1
RBD Mutations

| | | | | | SARS2- |
|---|---|---|---|---|---|
| | Beta | Gamma | Delta | Omicron | N501Y_{MA30} |
| | (SEQ | (SEQ | (SEQ | (SEQ | (SEQ |
| | ID NO) | ID NO) | ID NO) | ID NO) | ID NO) |
| G339 | — | — | — | G339D (22) | — |
| S371 | — | — | — | S371L (23) | — |
| S373 | — | — | — | S373P (24) | — |
| S375 | — | — | — | S375F (25) | — |
| K417* | K417N | K417T | — | K417N (26) | K417M (28) |
| | (26) | (27) | | | |
| N440* | — | — | — | N440K (29) | — |
| G446 | — | — | — | G446S (30) | — |
| L452 | — | — | L452R | — | — |
| | | | (31) | | |
| S477 | — | — | — | S477N (32) | |
| T478 | — | — | T478K | T478K (33) | — |
| | | | (33) | | |
| E484* | E484K | E484K | — | E484A (35) | E484K (34) |
| | (34) | (34) | | | |
| Q493* | — | — | — | Q493K (36) | Q493R (37) |
| G496 | — | — | — | G496S (38) | — |
| Q498 | — | — | — | Q498R (39) | Q498R (39) |
| N501* | N501Y | N501Y | — | N501Y (40) | N501Y (40) |
| | (40) | (40) | | | |
| Y505 | — | — | — | Y505H (41) | — |

*Mutations associated with antibody escape

Notably SEQ ID NO: 22-41 contain individual mutations identified in the S protein and these individual mutations may be combined to form novel S proteins that may be used to generate the vaccines and VLPs described herein. For example, the delta variant of the virus contains the mutations provided in SEQ ID NO: 31 and 33 in combination. Thus, these described combinations as well as new combinations are also provided herein. It is likely that new combinations of mutations in the S protein will continue to arise in the circulating virus population and the vaccines and VLPs described herein will need to take account of the circulating virus in order to maintain immunogenicity.

As used herein, “SARS-CoV-2 antigen” refers to a SARS-CoV-2 protein, a sequence at least 90% identical thereto, a fragment thereof, or combinations thereof that may be used to elicit an immune response in a subject. The SARS-CoV-2 antigen may be the SARS-CoV-2 S protein, the SARS-CoV-2 M protein, the SARS-CoV-2 E protein, the SARS-CoV-2 N protein, the SARS-CoV-2 S protein RBD, a mutated SARS-CoV-2 S protein RBD (SEQ ID NO: 22-41) a protein with a sequence at least 90%, 95%, 98%, or 99% sequence identity thereto, or combinations thereof. Notably SEQ ID NO: 22-41 contain individual mutations identified in the S protein and these individual mutations may be combined to form novel S proteins that may be used to generate the vaccines and VLPs described herein. For example, the delta variant of the virus contains the mutations provided in SEQ ID NO: 31 and 33 in combination. Thus, these described combinations as well as new combinations are also provided herein. It is likely that new combinations of mutations in the S protein will continue to arise in the circulating virus population and the vaccines and VLPs described herein will need to take account of the circulating virus in order to maintain immunogenicity.

As used herein, the phrases “% sequence identity,” “percent identity,” or “% identity” are used interchangeably and refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. A suite of commonly used and freely available sequence comparison algorithms is provided by the National Center for Biotechnology Information (NCBI) Basic Local Alignment Search Tool (BLAST® alignment tool), which is available from several sources, including the NCBI, Bethesda, Md., at its website. The BLAST® alignment tool software suite includes various sequence analysis programs including “blastp,” that is used to align a known amino acid sequence with other amino acids sequences from a variety of databases.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Polynucleotides encoding any of the SARS-CoV-2 antigens described herein are provided. As used herein, the terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of natural or synthetic origin (which may be single-stranded or double-stranded and may represent the sense or the antisense strand). The polynucleotides may be cDNA or genomic DNA.

Polynucleotides homologous to the polynucleotides described herein are also provided. Those of skill in the art understand the degeneracy of the genetic code and that a variety of polynucleotides can encode the same polypeptide. In some embodiments, the polynucleotides (i.e., polynucleotides encoding the SARS-CoV-2 antigens described herein) may be codon-optimized for expression in a particular cell including, without limitation, a plant cell, mammalian cell, insect cell, bacterial cell, or fungal cell. While particular polynucleotide sequences are disclosed herein, any polynucleotide sequences may be used which encodes a desired form of the polypeptides described herein. Thus non-naturally occurring sequences may be used. These may be desirable, for example, to enhance expression in heterologous expression systems of polypeptides or proteins. Computer programs for generating degenerate coding sequences are available and can be used for this purpose. Pencil, paper, the genetic code, and a human hand can also be used to generate degenerate coding sequences.

In another aspect of the present invention, constructs are provided. As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies.

The constructs provided herein may be prepared by methods available to those of skill in the art. Notably each of the constructs claimed are recombinant molecules and as such do not occur in nature. Generally, the nomenclature used herein and the laboratory procedures utilized in the present invention include molecular, biochemical, and recombinant DNA techniques that are well known and commonly employed in the art. Standard techniques available to those skilled in the art may be used for cloning, DNA and RNA isolation, amplification and purification. Such techniques are thoroughly explained in the literature. The constructs provided herein may include a promoter operably linked to any one of the polynucleotides described herein. As used herein, a polynucleotide is “operably connected” or “operably linked” when it is placed into a functional relationship with a second polynucleotide sequence.

As used herein, the terms “heterologous promoter,” “promoter,” “promoter region,” or “promoter sequence” refer generally to transcriptional regulatory regions of a gene, which may be found at the 5′ or 3′ side of a polynucleotides described herein, or within the coding region of said polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

Heterologous promoters useful in the practice of the present invention include, but are not limited to, constitutive, inducible, temporally-regulated, developmentally regulated, chemically regulated, tissue-preferred and tissue-specific promoters. The heterologous promoter may be a plant, animal, bacterial, fungal, or synthetic promoter. Suitable promoters are known and described in the art. Suitable promoters for expression in plants include, without limitation, the 35S promoter of the cauliflower mosaic virus, ubiquitin, tCUP cryptic constitutive promoter, the Rsyn7 promoter, pathogen-inducible promoters, the maize In2-2 promoter, the tobacco PR-1a promoter, glucocorticoid-inducible promoters, estrogen-inducible promoters and tetracycline-inducible and tetracycline-repressible promoters. Other promoters include the T3, T7 and SP6 promoter sequences, which are often used for in vitro transcription of RNA. In mammalian cells, typical promoters include, without limitation, promoters for Rous sarcoma virus (RSV), human immunodeficiency virus (HIV-1), cytomegalovirus (CMV), SV40 virus, and the like as well as the translational elongation factor EF-1α promoter or ubiquitin promoter. In some embodiments, the promoter is viral synthetic late promoter (SLP). In some embodiments, the SLP has the sequence of SEQ ID NO:5. Those of skill in the art are familiar with a wide variety of additional promoters for use in various cell types.

The constructs provided herein may include a translation enhancing element (TEE) operably linked to any one of the polynucleotides described herein. As used herein “translation enhancing elements (TEE),” refers to polynucleotide sequences that mediate cap-independent translation initiation. A TEE polynucleotide refers to both the RNA polynucleotide being translated and the DNA polynucleotide encoding said RNA polynucleotide. Identification of TEEs is described in US Publication No. 20130230884 and described by Wellensiek et al. (“Genome-wide profiling of cap-independent translation enhancing elements in the human genome,” Nat Methods, 2013, 10(8):747-750). Suitable TEEs are also described in US Publication No. 20140255990 and Wellensiek et al. (“A leader sequence capable of enhancing RNA expression and protein synthesis in mammalian cells,” Protein Sci., 2013, 22(10):1392-1398). In some embodiments, the TEE includes the sequence of SEQ ID NO:4. In some embodiments, the TEE includes the sequence of SEQ ID NO:10. In some embodiments, the TEE includes the sequence of SEQ ID NO:11. In some embodiments, the TEE includes the sequence SEQ ID NO:12. In some embodiments, a polynucleotide sequence may act as both a promoter and a TEE.

Vectors including any of the constructs or polynucleotides described herein are provided. The term “vector” is intended to refer to a polynucleotide capable of transporting another polynucleotide to which it has been linked. In some embodiments, the vector may be a “plasmid,” which refers to a circular double-stranded DNA loop into which additional DNA segments may be ligated. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome, such as some viral vectors or transposons. Viral genomes are also included as vectors, including vectors based on viral genomes. Vectors may carry genetic elements, such as those that confer resistance to certain drugs or chemicals.

In some embodiments, the vector is a geminiviral vector used to increase yield of proteins expressed in plant systems. Geminiviral vectors are based on the genome of bean yellow dwarf virus (BeYDV). The geminiviral vectors include the genes Rep and RepA, as well as geminiviral short and long intergenic regions, in cis allows for the genes of interest to be amplified once delivered to the plant. Geminiviral vectors are described in U.S. Pat. No. 10,125,373 and US Publication No. 20190336596, which are incorporated herein by reference. Geminiviral vectors may be used for expression of SARS-CoV-2 antigens in plants, in particular Nicotiana benthamiana. In some embodiments, the geminiviral vectors includes polynucleotides encoding SARS-CoV-2 antigens, said polynucleotides being codon optimized for expression in N. benthamiana.

In some aspects, the vector is a vaccinia virus expression vector based on the vaccinia virus genome. Vaccinia virus (VACV or VV) is a large, complex, enveloped virus belonging to the poxvirus family. It has a linear, double-stranded DNA genome of approximately 190 kb in length, which encodes for around 250 genes. The genome is surrounded by a lipoprotein core membrane. The poxviruses are the largest known DNA viruses and are distinguished from other viruses by their ability to replicate entirely in the cytoplasm of the host cell, outside of the nucleus. VV can accept as much as 25 kb of foreign DNA, making it useful for expressing large genes. Foreign genes are integrated stably into the viral genome, resulting in efficient gene expression. Other viral expression vectors for use in the present invention include, but are not limited to, certain highly attenuated, host-restricted, non- or poorly replicating poxvirus strains have been developed for use as substrates in recombinant vaccine development. These strains include the Orthopoxviruses, Modified Vaccinia Ankara (MVA) and NYVAC (derived from the Copenhagen vaccinia strain), and the Avipoxviruses, ALVAC and TROVAC (derived from canarypox and fowlpox viruses, respectively). In some embodiments, the viral expression vectors described herein may be modified to have one or more desirable properties.

In some embodiments, the viral expression vector is a NYVAC vector that has been modified to be replication-competent with improved T cell and antibody responses to the delivered antigen. As used herein “NYVAC-KC” refers to a NYVAC vector modified to include a polynucleotide encoding the C7L polypeptide (SEQ ID NO:2) adjacent to a polynucleotide encoding the K1L polypeptide (SEQ ID NO:3). Both C7L and K1L have been shown to be involved in defining the replication competence of the virus. The NYVAC-KC vector is described in further detail in U.S. Pat. No. 9,670,506, which is incorporated herein by reference in its entirety.

In some embodiments, vectors described herein include an internal ribosomal entry site (IRES). In some embodiments, vectors described herein include at least two IRES. In some embodiments, vectors described herein include a self-cleaving protein element. In some embodiments, vectors described herein include at least two self-cleaving protein elements.

In some aspects, provided herein are virus-like particles (VLPs) or recombinant immune complexes incorporating the SARS-CoV-2 antigens described herein. As used herein, “virus-like particles (VLPs)” refers to particles that include one or more viral proteins and mimics the structural of the native virus but lack the viral genome. In some embodiments, the VLP includes at least the S protein. In some embodiments, the VLP includes at least the M and E proteins. In some embodiments, the VLP includes at least the M, E, and S proteins. In some embodiments, the VLP includes the M, E, S, and N proteins.

In some embodiments, the VLP is an antigen presenting VLP in which a SARS-CoV-2 protein is attached to a protein scaffold. The antigen presenting VLP is produced as a fusion protein of the protein scaffold and a SARS-CoV-2 antigen as described herein. Suitable protein scaffolds for use in an antigen presenting VLP are known in the art. In some embodiments, the protein scaffold is Hepatitis B core antigen (HBc). In the antigen presenting VLP, the antigen may be bound to the major insertion region of HBc. In some embodiments, the antigen presenting VLP includes HBc and the SARS-CoV-2 S protein. In some embodiments, the antigen presenting VLP includes HBc and the SARS-CoV-2 S protein RBD.

As used herein, “recombinant immune complexes (RICs)” refers to a complex including a SARS-CoV-2 antigen, an immunoglobulin heavy chain, and an epitope tag that can bind to the immunoglobulin heavy chain. RICs mimic the antigen-antibody complexes formed during natural infections to enhance immune responses to the antigen. In certain embodiments, the RIC further comprises an immunoglobulin light chain. Thus, in some aspects, the RIC comprises a standard antibody (two heavy chains and two light chains joined to form a “Y” shaped molecule), an antigen, and an epitope tag that is recognized by the antibody. The antibody binds to the epitope tags on other antibody fusions and forms a complex. In some embodiments, the RIC comprises human IgG 6D8 heavy chain, and the epitope tag is ebola glycoprotein epitope 6D8 (SEQ ID NO:13). A universal RIC platform is described in detail in Kim et al. (“Novel vaccine approach for dengue infection based on recombinant immune complex universal platform,” Vaccine, 2015, 33(15):1830-1838) and US Publication No. 2019/0336596, each of which is incorporated herein by reference.

RICs described herein include conventional RICs where the target antigen is linked to the C-terminus of the immunoglobulin heavy chain and the epitope tag is linked to the other end of the target antigen (also referred to herein as “C-RIC”). The recombinant immune complex is produced by fusing a target antigen to the C-terminus of the heavy chain of an immunoglobulin that binds specifically to the antigen, wherein the co-expression of this fusion protein with the light chain of the antibody produces a fully formed immunoglobulin that is self-reactive, and results in the creation of an immune complex due to the bivalent binding capacity of the immunoglobulin. However, antigens with inaccessible N-termini cannot be easily used in the RIC platform without disrupting native antigenic conformation. Also described herein are RICs where the target antigen is linked to the N-terminus of the immunoglobulin heavy chain and the epitope tag is linked to C-terminus of the immunoglobulin heavy chain (also referred to herein as “N-RIC”).

In certain embodiment of an expression vector encoding RICs, the expression vector comprises an expression cassette encoding the immunoglobulin heavy chain, the target antigen, and the epitope tag. In some aspects, the expression vector further comprises a second expression cassette encoding the immunoglobulin light chain. VLPs and RICs as described herein may be produced using any suitable method known in the art.

In some aspects, described herein are methods for producing a VLP or an MC in a plant. Plant based systems for protein expression are known and described in the art. See for example, U.S. Pat. No. 10,125,373, incorporated herein by reference. In general, a plant is transfected with a construct or vector as described herein encoding a SARS-CoV-2 antigen. In some embodiments, the vector is a geminiviral vector. The plant may be transfected using Agrobacterium mediated transformation, particle bombardment, or another suitable method known in the art. Transfection of the plant also includes transfection of a portion or part of the plant including, but not limited to, a part of a root, leaf, stem, seed, pod, flower, cell, tissue plant germplasm, asexual propagate, or any progeny thereof. In some embodiments, the plant is N. benthamiana.

Vaccine compositions including the SARS-CoV-2 antigens, VLPs, or RICs described herein are also provided. As used herein “vaccine” refers to a composition that includes an antigen. Vaccine may also include a biological preparation that improves immunity to a particular disease. A vaccine may typically contain an agent, referred to as an antigen, that resembles a disease-causing microorganism, in this case SARS-CoV-2, and the agent may often be made from weakened or killed forms of the microbe, its toxins or one of its surface proteins. The antigen may stimulate the body's immune system to recognize the agent as foreign, destroy it, and “remember” it, so that the immune system can more easily recognize and destroy any of these microorganisms that it later encounters.

Vaccines may be prophylactic, e.g., to prevent or ameliorate the effects of a future infection by any natural or “wild” pathogen, or therapeutic, e.g., to treat the disease. Administration of the vaccine to a subject results in an immune response, generally against one or more specific diseases. The amount of a vaccine that is therapeutically effective may vary depending on the particular virus used, or the condition of the patient, and may be determined by a physician. The vaccine may be introduced directly into the subject by the subcutaneous, oral, oronasal, or intranasal routes of administration.

The vaccine compositions described herein also include a suitable carrier or vehicle for delivery. As used herein, the term “carrier” refers to a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, may be found in the U.S. Pharmacopeia National Formulary, 1857-1859, (1990).

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

In another embodiment, the present formulation may also comprise other suitable agents such as a stabilizing delivery vehicle, carrier, support or complex-forming species. The coordinate administration methods and combinatorial formulations of the instant invention may optionally incorporate effective carriers, processing agents, or delivery vehicles, to provide improved formulations for delivery of the SARS-CoV-2 antigens, VLPs, or RICs described herein.

The vaccine formulation may additionally include a biologically acceptable buffer to maintain a pH close to neutral (7.0-7.3). Such buffers preferably used are typically phosphates, carboxylates, and bicarbonates. More preferred buffering agents are sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the vaccine formulation, more preferably about 0.001-1% (w/v). Other excipients, if desired, may be included as part of the final vaccine formulation.

In some embodiments, the present formulation may also comprise an adjuvant. An adjuvant is a substance or combination of substances that is used to increase the efficacy or potency of the formulation or modulates the immune response to a vaccine. An adjuvant may accelerate, prolong or enhance antigen-specific immune responses when used in combination with an antigen. An adjuvant may be an inorganic compound such as potassium alum, aluminum hydroxide, aluminum phosphate or calcium phosphate hydroxide. An adjuvant may also be an oil such as paraffin oil or propolis, a bacterial product such as killed Bordetella pertussis or Mycobacterium bovis or their toxoids, monophosphoryl lipid A or detoxified Salmonella minnesota lipopolysaccharide. An adjuvant may also be derived from plants, such as saponins from Quillaja (QS-21), soybean or Polygala senega. Cytokines such as IL-1, IL-2 or IL-12 may also act as adjuvants. Adjuvants may also include Freund's complete or incomplete adjuvant or squalene including AS03 or MF59. The remainder of the vaccine formulation may be an acceptable diluent, to 100%, including water. The vaccine formulation may also be formulated as part of a water-in-oil, or oil-in-water emulsion.

The vaccine formulation may be separated into vials or other suitable containers. The vaccine formulation herein described may then be packaged in individual or multi-dose ampoules, or be subsequently lyophilized (freeze-dried) before packaging in individual or multi-dose ampoules. The vaccine formulation herein contemplated also includes the lyophilized version. The lyophilized vaccine formulation may be stored for extended periods of time without loss of viability at ambient temperatures. The lyophilized vaccine may be reconstituted by the end user and administered to a patient.

The term “lyophilization” or “lyophilized,” as used herein, refers to freezing of a material at low temperature followed by dehydration by sublimation, usually under a high vacuum. Lyophilization is also known as freeze drying. Many techniques of freezing are known in the art of lyophilization such as tray-freezing, shelf-freezing, spray-freezing, shell-freezing and liquid nitrogen immersion. Each technique will result in a different rate of freezing. Shell-freezing may be automated or manual. For example, flasks can be automatically rotated by motor driven rollers in a refrigerated bath containing alcohol, acetone, liquid nitrogen, or any other appropriate fluid. A thin coating of product is evenly frozen around the inside “shell” of a flask, permitting a greater volume of material to be safely processed during each freeze-drying run. Tray-freezing may be performed by, for example, placing the samples in lyophilizer, equilibrating 1 hr at a shelf temperature of 0° C., then cooling the shelves at 0.5° C./min to −40° C. Spray-freezing, for example, may be performed by spray-freezing into liquid, dropping by ˜20 μl droplets into liquid N2, spray-freezing into vapor over liquid, or by other techniques known in the art.

Methods of inducing an immune response in a subject art also provided. A vaccine composition as described herein and including a SARS-CoV-2 antigen, VLP, or RIC as described herein is administered to subject to induce an immune response. Following administration, the immune response of the subject may be tested using methods known in the art.

To vaccinate a subject, a therapeutically effective amount of a vaccine composition described herein is administered to the subject. The therapeutically effective amount of vaccine may typically be one or more doses, preferably in the range of about 0.01-10 mL, most preferably 0.1-1 mL, containing 1-500 micrograms, most preferably 1-100 micrograms of vaccine formulation/dose. The therapeutically effective amount may also depend on the vaccination species. For example, for smaller animals such as mice, a preferred dosage may be about 0.01-1 mL of a 1-50 microgram solution of antigen. For a human patient, a preferred dosage may be about 0.1-1 mL of a 1-50 microgram solution of antigen. The therapeutically effective amount may also depend on other conditions including characteristics of the patient (age, body weight, gender, health condition, etc.), and others.

The term “administration,” as used herein, refers to the introduction of a substance, such as a vaccine, into a subject's body. The administration, e.g., parenteral administration, may include subcutaneous administration, intramuscular administration, transcutaneous administration, intradermal administration, intraperitoneal administration, intraocular administration, intranasal administration, oral administration and intravenous administration.

The vaccine or the composition according to the invention may be administered to an individual according to methods known in the art. Such methods comprise application e.g. parenterally, such as through all routes of injection into or through the skin: e.g. intramuscular, intravenous, intraperitoneal, intradermal, mucosal, submucosal, or subcutaneous. Also, the vaccine may be applied by topical application as a drop, spray, gel or ointment to the mucosal epithelium of the eye, nose, mouth, anus, or vagina, or onto the epidermis of the outer skin at any part of the body.

Other possible routes of application are by spray, aerosol, or powder application through inhalation via the respiratory tract. In this last case, the particle size that is used will determine how deep the particles will penetrate into the respiratory tract.

Alternatively, application may be via the alimentary route, by combining with the food, feed or drinking water e.g. as a powder, a liquid, or tablet, or by administration directly into the mouth as a: liquid, a gel, a tablet, or a capsule, or to the anus as a suppository.

The present disclosure is generally applied to mammals, including but not limited to humans, cows, horses, sheep, pigs, goats, rabbits, dogs, cats, bats, mice, mink, deer, and rats. In some embodiments, the present disclosure can be applied to birds. In certain embodiments, non-human mammals, such as mice and rats, may also be used for the purpose of demonstration. One may use the present invention for veterinary purpose. For example, one may wish to treat commercially important farm animals, such as cows, horses, pigs, rabbits, goats, sheep, and birds, such as chickens. One may also wish to treat companion animals, such as cats and dogs.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

The present invention has been described in terms of one or more preferred embodiments, and it should be appreciated that many equivalents, alternatives, variations, and modifications, aside from those expressly stated, are possible and within the scope of the invention.

### EXAMPLES

The following example describes methods for making SARS-CoV-2 antigen VLPs and immune complexes in plants and use of said SARS-CoV-2 antigen VLPs and immune complexes.

### Example 1: Plant VLPS

Production of biologics in plants has made great strides during the past decade, becoming FDA approved. Innovations in expression vectors for transient expression have produced new plant expression systems with the flexibility and speed that cannot be matched by those based on mammalian or insect cell culture. For example, plant transient expression with our “deconstructed” plant viral vectors allows the production of up to 5 mg of VLP vaccines per gram of leaf fresh weight within 4-10 days of vector inoculation. The rapid and high-level protein production capability of transient expression systems make them the optimal system to quickly develop and produce vaccines against viruses such as SARS-coronavirus-2 that have sudden and unpredictable outbreaks. VLPs can display various heterologous antigens on their surface. Results have shown that these chimeric VLPs are stable, highly immunogenic, and can be produced robustly in plants. Recently, we have developed a system in which plant-produced VLPs can deliver the VLP-coding plasmid and replicate one round in the host cell, combining the advantages of both VLP and DNA-based vaccines. Furthermore, the replication process mimics viral infection and increases cellular immune response to the antigen. The efficient production of enveloped VLPs have been also demonstrated in plants. Of note, plant-produced hemagglutinin influenza VLPs elicited greater T cell responses, as well as antibody responses than an inactivated influenza vaccine due to its unique plant lipids in the envelope.

Immune complexes, defined as antibodies bound to their cognate antigens, have long been known to enhance immune responses in animal models. We and others have developed recombinant immune complexes (RIC), which consist of an antibody fused to its cognate antigen and are able to form large antigen-antibody complexes that mimic those found during natural infection. This complex formation results in a number of benefits including direct activation of antigen presenting cells via cross-linked Fc-gamma receptors, enhancement of antigen presentation to B-cells, effective stimulation of immune responses through high avidity C1q binding, and increased T-cell activation. Recently, we demonstrated that when VLP and RIC are co-delivered, a synergistic enhancement of antigen-specific antibody titers and virus neutralization is achieved.

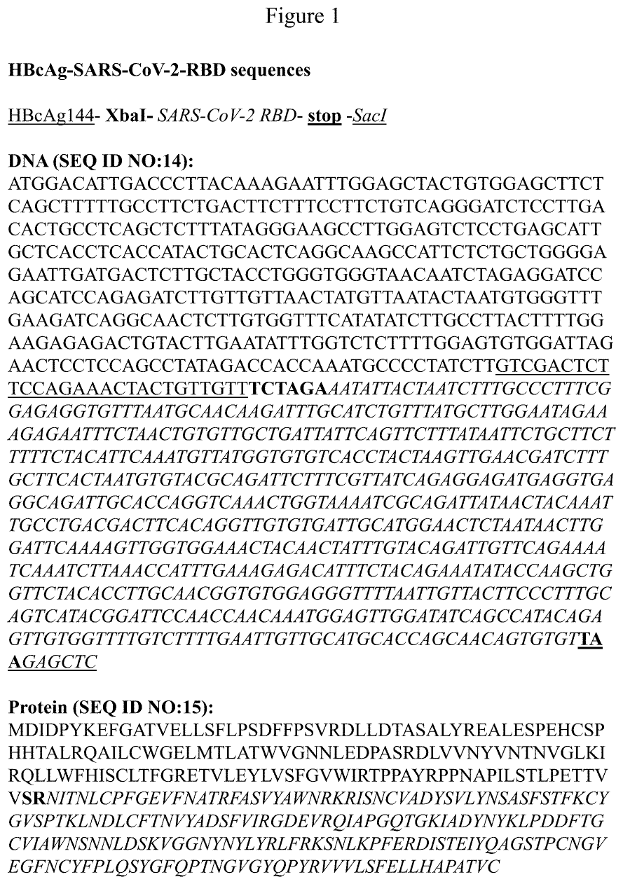

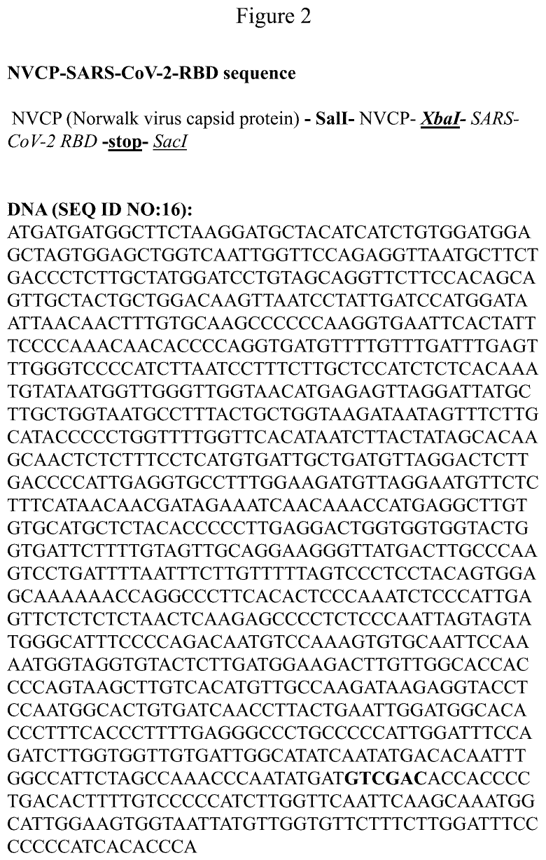

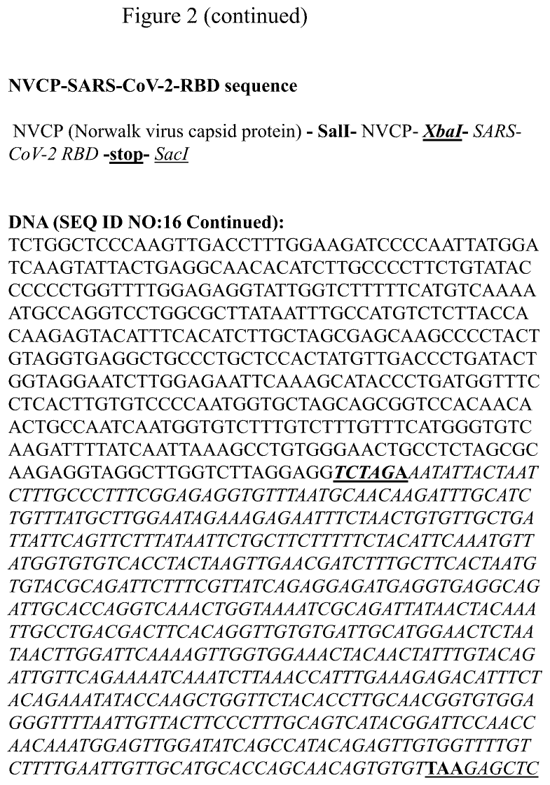

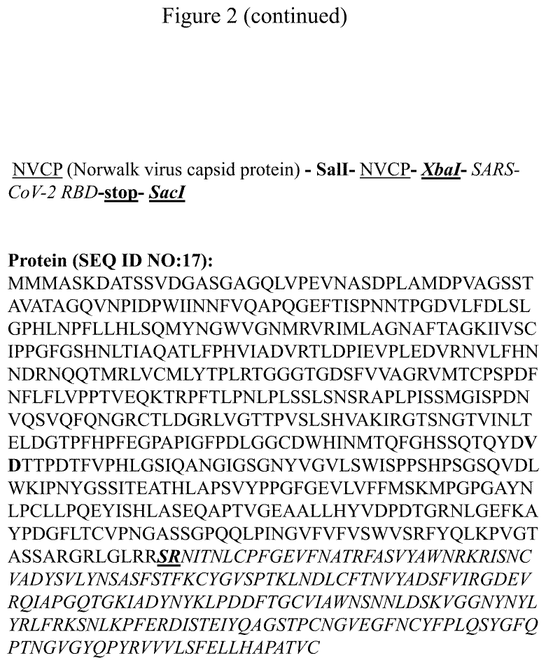

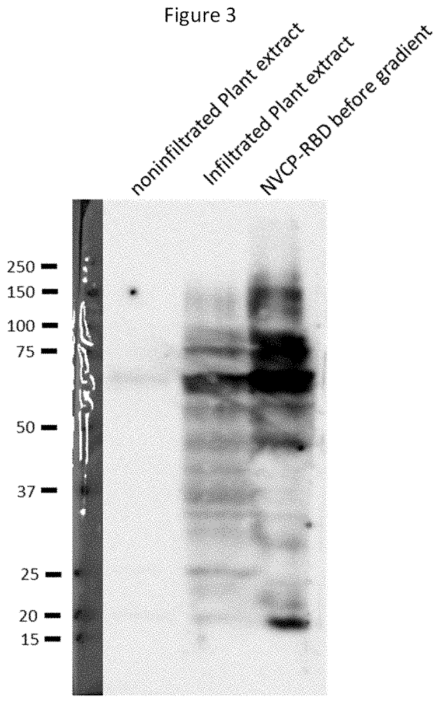

Production of S antigen presenting VLPs in plants. Hepatitis B core (HBc)-based VLPs are known as one of the most immunogenic antigens and carrier vehicles in immunization strategies. Our published results have shown that HBc VLPs that display the domain III of Zika virus or West Nile virus envelope protein can be produced in large quantities in Nicotiana benthamiana plants and elicited potent neutralizing antibody and cell-mediated immune responses and protected mice from lethal flavivirus challenges. HBc VLPs have also shown the capacity to display proteins of various sizes, from small peptides to large whole proteins. The coding sequence of the receptor-binding domain (RBD) domain of the S protein was inserted in frame into coding sequence of the major insertion region (MIR) on the HBc protein gene, forming HBcAg-SARS-CoV-2-RBD (FIG. 1, SEQ ID NO: 14), for producing chimeric VLPs. Additionally, the RBD domain of the S protein was inserted into the Norwalk virus capsid protein (NVCP; FIG. 2, SEQ ID NO: 16). The RDB gene constructs were cloned into a geminiviral vector and then infiltrated into leaves of N. benthamiana to rapidly produce and screen the VLP candidate. In FIG. 3, we demonstrate that this method can be used to produce NVCP-SARS-CoV-2-RBD in plants using a western blot.

Based on our previous experience, milligrams of VLPs can be produced and purified following published procedures within a week of gene infiltration. The unique rolling circle replication of geminiviral vector would allow the VLP replicons to be packaged inside the VLPs and direct one round of replication in the host cell. The VLPs will be characterized by SDS-PAGE, ELISA, electron microscopy as previously described and by RT-PCR for assessing the copy numbers of HBC-RBD replicons inside the VLPs, as well as by vaccinating a murine model of infection.

Evaluation of immune response following immunization in murine model. Balb/c mice will be immunized systemically (i.m.) in the absence or presence of alum adjuvant with selected VLPs or RICs prepared. We plan to use alum for VLPs and RICs, because it has been approved for the use as an adjuvant in injectable human vaccines (such as recombinant HBsAg vaccines) and can induce a strong Th2-biased antibody response. Immunity induced by replicating pox vectors is usually not enhanced by the presence of an adjuvant (unpublished observations). Serum samples will be collected and analyzed for the presence of spike and RBD specific serum IgG by antigen-specific ELISAs. Serum IgG1 and IgG2a will also be analyzed to determine the type (Th1 vs Th2) of the immune response to individual immunogen/adjuvant combinations. Finally, serum samples will be assayed for neutralizing capacity using VSV pseudotyped with SARS-Coronavirus-2 S protein. Balb/c mice will also be immunized intranasally in the absence or presence of synthetic CpG oligodeoxynucleotides (ODN) with selected VLPs or RICs. If weak mucosal immune responses are observed following intranasal immunization only, we will try an intranasal priming/systemic boosting strategy which has been shown to be effective in augmenting sIgA production.

Neutralization assays using SARS-Coronavirus-2 neutralizing capacity of the serum and mucosal antibodies will be analyzed using VSV pseudotyped with SARS-Coronavirus-2 S protein.

Production of soluble S antigen and S RBD-presenting RIC in plants. The soluble S antigen will be produced by fusing the coding sequence of the S ectodomain to a His6-tag and transiently expressed in plants as described above for VLPs. A universal RIC platform has already been established to produce RIC without the need of finding specific antibody-antigen pair. To express RBD-specific RIC in plants, the coding sequence of S RBD domain will be inserted between the C-terminus of 6D8 antibody heavy chain and the 6D8 epitope in our established RIC cassette inside the geminiviral vector. One week after gene introduction into leaves, milligrams of soluble S protein and RBD-RIC will be produced and purified by Ni column and protein A chromatography as described previously. The S protein and RBD-RIC will be characterized by SDS-PAGE, ELISA, and size-exclusion chromatograph for RIC formation as previously described. Since a synergistic enhancement of the immune response has been demonstrated when RIC and VLP are co-delivered, soluble S, RBD-RIC and HBc-RBD VLP+RBD-RIC will be used in vaccinating in a murine model as described above.

Production of enveloped SARS-coronavirus-2 VLPs. Recombinant expression of the major structural proteins for many viruses results in VLP formation. Co-expression of only M and E is sufficient for VLP assembly, but S is incorporated when it is also expressed. In some cases, expression of the N gene enhances VLP production. Different protein combinations have been reported for production of SARS-CoV VLPs. VLPs are efficiently produced when M, E, and S are co-expressed in insect cells. Co-expression of the M protein with N or E proteins was reported to be sufficient for VLP assembly in mammalian cells, whereas another group showed that M must be expressed with N for efficient production and release of VLPs and that the S protein is incorporated when included. Thus, the requirements for SARS-CoV VLP production are still not absolutely clear. The result from experiments for SARS-CoV VLP production will inform which proteins among E, M, S and N proteins are essential to form stable VLPs with the optimal immunogenicity. Based on such results, genes for the essential proteins will be individually cloned into geminiviral vectors and co-expressed in plants to promote VLP formation. Previous results have demonstrated the efficient production and purification of enveloped VLPs from plants. The production of clarified plant extract will follow the standard protocols and VLP will be purified from the extract following the protocol established in SARS-CoV VLP production experiments and characterized. The immunogenicity of plant-produced SARS-Coronavirus-2 VLP will be examined in vaccinating murine model as described above. Due to the unique plant lipids in the VLP, we expect a higher T and B cell response by this antigen.

We expect that the proposed vaccine candidates will be quickly produced and purified from plants in sufficient quantities for characterization and immunological studies. Due to the replicating nature of the chimeric VLP and the presence of plant lipid on the enveloped VLPs, we expect the VLP, RIC-based and the combination of both candidates will elicit potent and neutralizing immune response against SARS-Coronavirus-2. Although VLPs have been shown to be an excellent oral immunogen in the absence of adjuvant, it is possible that when delivered without adjuvant, VLPs may have lower immunogenicity. In this case, we will test various adjuvants to enhance their immunity.

### Example 2: Vaccination and Challenge in Mice

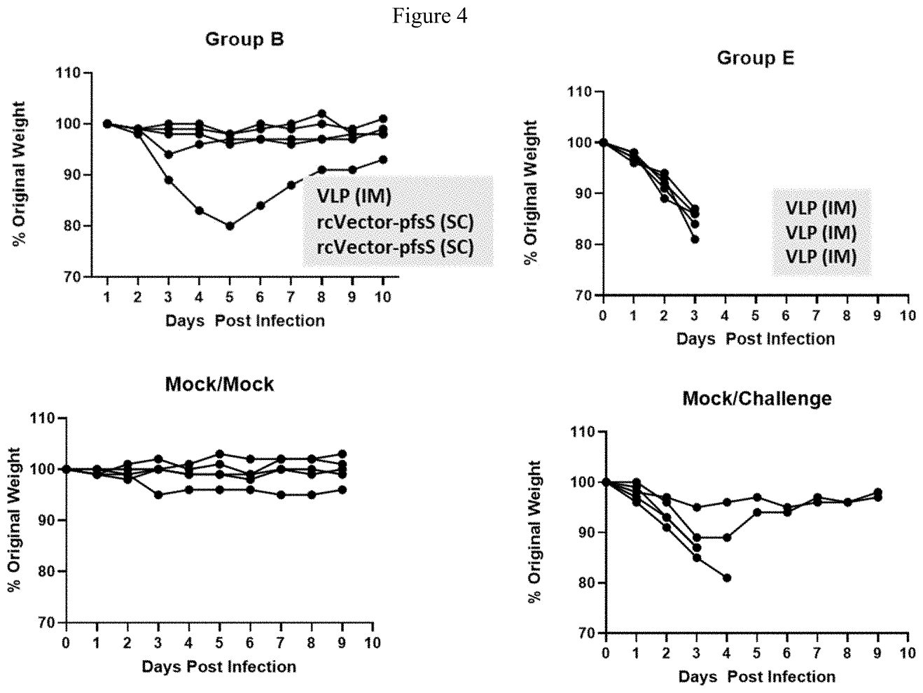

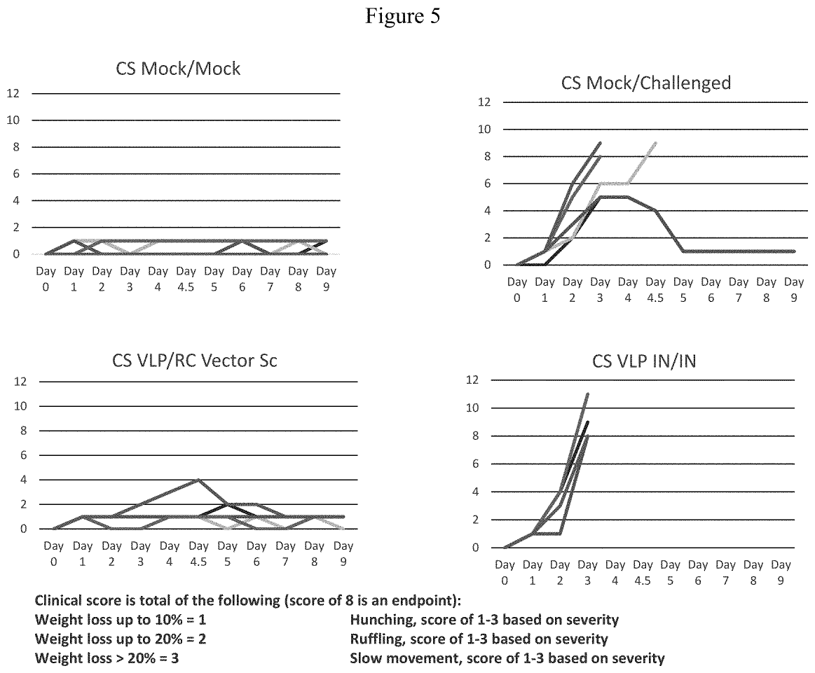

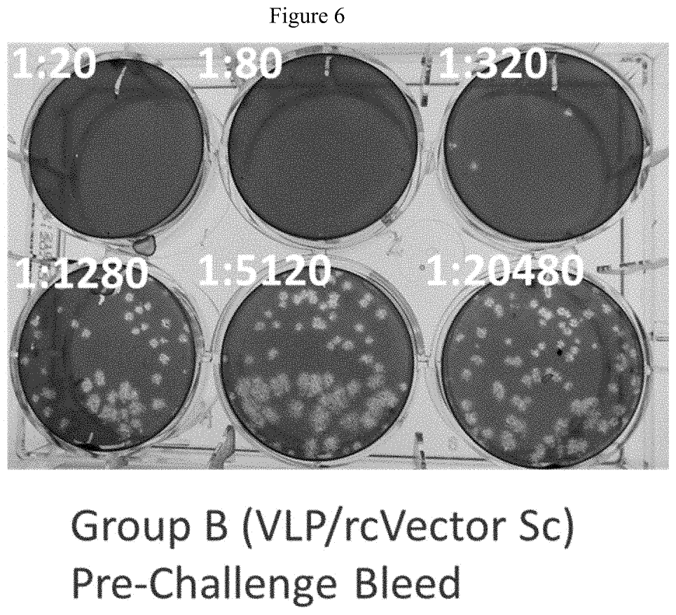

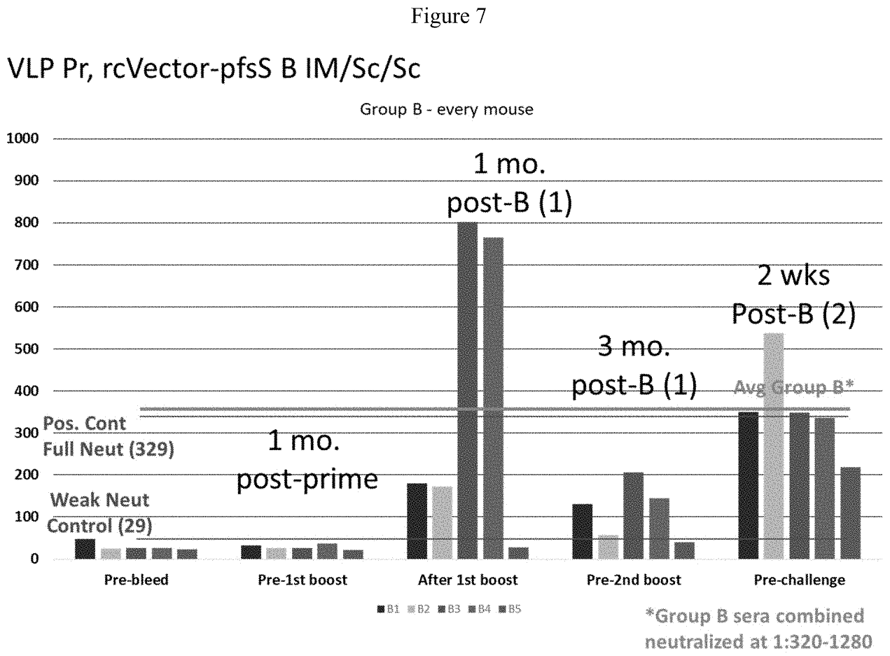

We next tested the ability of our candidate replication-competent vaccine vector to protect mice from challenge with a mouse-adapted SARS-CoV-2 virus. Mice in group B were vaccinated with a prime of a plant-derived VLP intramuscularly and two boosts of the replication-competent vaccine sub-cutaneously. All B mice were protected from viral challenge, as evidenced by the fact that their body weights remained stable for at least 10 days post infection (FIG. 4). In contrast, the mice in groups E, which were vaccinated with a prime and two boosts of the VLP intramuscularly did not show any protection (FIG. 4). The clincal scores that were used to determine study endpoints (i.e., when to euthanize the mice) reveal a huge divergence between the outcomes for protected and non-protected mice (FIG. 5). The ability of the replication-competant vaccine to generate SARS-CoV-2 neutralizing antibodies was demonstrated by assaying for neutralizing activity in serum samples collected from the mice at five time points: before prime, one month after prime (prior to the 1^{st} boost), one month after the 1^{st} boost, three months after the 1^{st} boost (prior to the 2^{nd} boost), and two weeks after the 2^{nd} boost (FIG. 7) shows significant neutralizing antibody responses.

### Embodiments of the Invention

Embodiment 1: A geminiviral vector comprising a polynucleotide encoding a Severe Acute Respiratory Syndrome Coronavirus 2 (SARS-CoV-2) antigen; and a polynucleotide encoding Hepatitis B core antigen (HBc) or the Norwalk capsid protein.

Embodiment 2: The vector of embodiment 1, wherein the geminiviral vector is based on bean yellow dwarf virus (BeYDV) genome.

Embodiment 3: The vector of embodiment 1 or 2, wherein the polynucleotide encoding a SARS-CoV-2 antigen is codon optimized for Nicotiana benthamiana.

Embodiment 4: The vector of any of embodiments 1-3, wherein the SARS-CoV-2 antigen is selected from the group consisting of SEQ ID NO:1, SEQ ID NO: 18, SEQ ID NO:6, SEQ ID NO:7, SEQ ID NO:8, SEQ ID NO:9, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, sequences at least 90% identical thereto, and combinations thereof.

Embodiment 5: A method for producing a SARS-CoV-2 VLP comprising expressing in a plant the vector of any of embodiments 1-4; and purifying the SARS-CoV-2 VLP from the plant.

Embodiment 6: The method of embodiment 5, wherein the plant is Nicotiana benthamiana. Embodiment 7: The method of embodiment 5 or 6, wherein the vector is transfected into a leaf of the plant.

Embodiment 8: The method of any of embodiments 5-7, comprising expressing in the plant at least two vectors each encoding a different SARS-CoV-2 antigen.

Embodiment 9: The method of any of embodiments 5-8, comprising expressing in the plant at least three vectors each encoding a different SARS-CoV-2 antigen.

Embodiment 10: The method of any of embodiments 5-9, wherein the VLP is purified using chromatography.

Embodiment 11: The method of any of embodiments 5-10, wherein the plant is transfected with the vector and the VLP is purified at least 3 days after transfection.

Embodiment 12: A SARS-CoV-2 VLP produced by the method of any of embodiments 5-11.

Embodiment 13: The SARS-CoV-2 VLP of embodiment 12, wherein the VLP comprises a SARS-CoV-2 antigen selected from the group consisting of SEQ ID NO:1, SEQ ID NO: 18, SEQ ID NO:6, SEQ ID NO:7, SEQ ID NO:8, SEQ ID NO:9, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, sequences at least 90% identical thereto, and combinations thereof.

Embodiment 14: A vaccine composition comprising the SARS-CoV-2 VLP of embodiment 12 or 13 and a pharmaceutically acceptable carrier.

Embodiment 15: The vaccine composition of embodiment 14, additionally comprising an adjuvant.

Embodiment 16: A method of inducing an immune response in a subject against a SARS-CoV-2 antigen comprising administering an effective amount of the vaccine composition of embodiment 14 or 15 to the subject.

Embodiment 17: The method of embodiment 16, wherein the SARS-CoV-2 antigen is selected from the group consisting of SEQ ID NO:1, SEQ ID NO: 18, SEQ ID NO:6, SEQ ID NO:7, SEQ ID NO:8, SEQ ID NO:9, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, sequences at least 90% identical thereto, and combinations thereof.

Embodiment 18: The method of embodiment 16 or 17, wherein the SARS-CoV-2 antigen is SEQ ID NO:1 or a sequence 90% identical thereto.

Embodiment 19: The method of any of embodiments 16-18, wherein the subject is a human.

Embodiment 20: A vector comprising a first polynucleotide encoding a SARS-CoV-2 antigen; a second polynucleotide encoding an immunoglobulin heavy chain; and a third polynucleotide encoding an epitope tag that binds to the immunoglobulin heavy chain, wherein the first, second, and third polynucleotides are arranged in the vector such that, when expressed from the vector, the SARS-CoV-2 antigen is between the C-terminus of the immunoglobulin heavy chain and the N-terminus of the epitope tag to form a recombinant immune complex (MC).

Embodiment 21: The vector of embodiment 20, wherein the vector is a geminiviral vector.

Embodiment 22: The vector of embodiment 21, wherein the geminiviral vector is based on bean yellow dwarf virus (BeYDV) genome.

Embodiment 23: The vector of any of embodiments 20-22, wherein the polynucleotide encoding a SARS-CoV-2 antigen is codon optimized for Nicotiana benthamiana.

Embodiment 24: The vector of any of embodiments 20-23, wherein the SARS-CoV-2 antigen is selected from the group consisting of SEQ ID NO:1, SEQ ID NO: 18, SEQ ID NO:6, SEQ ID NO:7, SEQ ID NO:8, SEQ ID NO:9, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, sequences at least 90% identical thereto, and combinations thereof.

Embodiment 25: A method for producing a SARS-CoV-2 RIC comprising expressing in a plant the vector of any of embodiments 20-24; and purifying the SARS-CoV-2 RIC from the plant.

Embodiment 26: The method of embodiment 25, wherein the plant is Nicotiana benthamiana.

Embodiment 27: The method of embodiment 25 or 26, wherein the vector is transfected into a leaf of the plant.

Embodiment 28: A SARS-CoV-2 RIC produced by the method of any of embodiments 25-27.

Embodiment 29: The SARS-CoV-2 RIC of embodiment 28 comprising the SARS-CoV-2 antigen positioned between the C-terminus of the immunoglobulin heavy chain and the N-terminus of the epitope tag.

Embodiment 30: The SARS-CoV-2 RIC of embodiment 28 or 29, wherein the SARS-CoV-2 antigen is selected from the group consisting of SEQ ID NO:1, SEQ ID NO: 18, SEQ ID NO:6, SEQ ID NO:7, SEQ ID NO:8, SEQ ID NO:9, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, sequences at least 90% identical thereto, and combinations thereof.

Embodiment 31: A vaccine composition comprising the SARS-CoV-2 RIC of any of embodiments 28-30 and a pharmaceutically acceptable carrier.

Embodiment 32: The vaccine composition of embodiment 31, additionally comprising an adjuvant.

Embodiment 33: A method of inducing an immune response in a subject against a SARS-CoV-2 antigen comprising administering an effective amount of the vaccine composition of embodiment 31 or 32 to the subject.

Embodiment 34: The method of embodiment 33, wherein the SARS-CoV-2 antigen is selected from the group consisting of SEQ ID NO:1, SEQ ID NO: 18, SEQ ID NO:6, SEQ ID NO:7, SEQ ID NO:8, SEQ ID NO:9, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, sequences at least 90% identical thereto, and combinations thereof.

Embodiment 35: The method of embodiment 33 or 34, wherein the SARS-CoV-2 antigen is SEQ ID NO:1 or a sequence at least 90% identical thereto.

Embodiment 36: The method of any of embodiments 33-35, wherein the subject is a human.

We claim:

1. A geminiviral vector comprising
a polynucleotide encoding a severe acute respiratory syndrome coronavirus 2 (SARS-CoV-2) antigen; and
a polynucleotide encoding Hepatitis B core antigen (HBc) or the Norwalk capsid protein.

2. The vector of claim 1, wherein the geminiviral vector is based on bean yellow dwarf virus (BeYDV) genome.

3. The vector of claim 1, wherein the polynucleotide encoding a SARS-CoV-2 antigen is codon optimized for Nicotiana benthamiana.

4. The vector of claim 1, wherein the SARS-CoV-2 antigen is selected from the group consisting SEQ ID NO:1, SEQ ID NO: 18, SEQ ID NO:6, SEQ ID NO:7, SEQ ID NO:8, SEQ ID NO:9, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, sequences at least 90% identical thereto, and combinations thereof.

5. A method for producing a SARS-CoV-2 VLP comprising
expressing in a plant the vector of claim 1; and
purifying the SARS-CoV-2 VLP from the plant.

6. The method of claim 5, wherein the plant is Nicotiana benthamiana.

7. The method of claim 5, comprising expressing in the plant at least two vectors each encoding a different SARS-CoV-2 antigen.

8. A SARS-CoV-2 VLP produced by the method of claim 5.

9. The SARS-CoV-2 VLP of claim 8, wherein the VLP comprises a SARS-CoV-2 antigen selected from the group consisting of SEQ ID NO:1, SEQ ID NO: 18, SEQ ID NO:6, SEQ ID NO:7, SEQ ID NO:8, SEQ ID NO:9, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, sequences at least 90% identical thereto, and combinations thereof.

10. A vaccine composition comprising the SARS-CoV-2 VLP of claim 8 and a pharmaceutically acceptable carrier.

11. A method of inducing an immune response in a subject against a SARS-CoV-2 antigen comprising administering an effective amount of the vaccine composition of claim 10 to the subject.

12. A vector comprising
a first polynucleotide encoding a SARS-CoV-2 antigen;
a second polynucleotide encoding an immunoglobulin heavy chain; and
a third polynucleotide encoding an epitope tag that binds to the immunoglobulin heavy chain, wherein the first, second, and third polynucleotides are arranged in the vector such that, when expressed from the vector, the SARS-CoV-2 antigen is between the C-terminus of the immunoglobulin heavy chain and the N-terminus of the epitope tag to form a recombinant immune complex (MC).

13. The vector of claim 12, wherein the vector is a geminiviral vector.

14. The vector of claim 12, wherein the polynucleotide encoding a SARS-CoV-2 antigen is codon optimized for Nicotiana benthamiana.

15. The vector of claim 12, wherein the SARS-CoV-2 antigen is selected from the group consisting of SEQ ID NO:1, SEQ ID NO:18, SEQ ID NO:6, SEQ ID NO:7, SEQ ID NO:8, SEQ ID NO:9, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, sequences at least 90% identical thereto, and combinations thereof.

16. A method for producing a SARS-CoV-2 RIC comprising
expressing in a plant the vector of claim 12; and
purifying the SARS-CoV-2 RIC from the plant.

17. The method of claim 16, wherein the plant is Nicotiana benthamiana.

18. A SARS-CoV-2 RIC produced by the method of claim 16.

19. A vaccine composition comprising the SARS-CoV-2 RIC of claim 18 and a pharmaceutically acceptable carrier.

20. A method of inducing an immune response in a subject against a SARS-CoV-2 antigen comprising administering an effective amount of the vaccine composition of claim 19 to the subject.


SEQUENCE LISTING (41 sequences)

SEQ ID NO: 1 - protein, 1273 aa, Severe Acute Respiratory Syndrome Coronaviruses 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 150 aa, Artificial Sequence
  Synthetic- C7L polypeptide
MGIQHEFDIIINGDIALRNLQLHKGDNYGCKLKIISNDYKKLKFRFIIRPDWSEIDEVKGLTVFANNYAVKVNKVDDTFYYVIYEAVIHLYNKKTEILIYSDDENELFKHYYPYISLNMISKKYKVKEENYSSPYIEHPLIPYRDYESMD

SEQ ID NO: 3 - protein, 284 aa, Artificial Sequence
  Synthetic- K1L polypeptide
MDLSRINTWKSKQLKSFLSSKDTFKADVHGHSALYYAIADNNVRLVCTLLNAGALKNLLENEFPLHQAATLEDTKIVKILLFSGMDDSQFDDKGNTALYYAVDSGNMQTVKLFVKKNWRLMFYGKTGWKTSFYHAVMLNDVSIVSYFLSEIPSTFDLAILLSCIHTTIKNGHVDMMILLLDYMTSTNTNNSLLFIPDIKLAIDNKDIEMLQALFKYDINIYSVNLENVLLDDAEITKMIIEKHVEYKSDSYTKDLDIVKNNKLDEIISKNKELRLMYVNCVKKN

SEQ ID NO: 4 - DNA, 11 bp, Artificial Sequence
  Synthetic- TEE
aaaactgctaa

SEQ ID NO: 5 - DNA, 26 bp, Artificial Sequence
  Synthetic- SLP
catattgaagagacagagtgatatat

SEQ ID NO: 6 - protein, 193 aa, Severe Acute Respiratory Syndrome Coronaviruses 2
  MISC_FEATURE (1)..(193): Receptor Binding Domain (RBD) 318-510 of SEQ ID NO1
FRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 7 - protein, 222 aa, Severe Acute Respiratory Syndrome Coronaviruses 2
  MISC_FEATURE (1)..(222): Membrane Protein
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 8 - protein, 75 aa, Severe Acute Respiratory Syndrome Coronaviruses 2
  MISC_FEATURE (1)..(75): Envelope protein
MYSFVSEETGTLIVNSVLLFLAFVVFLLVTLAILTALRLCAYCCNIVNVSLVKPSFYVYSRVKNLNSSRVPDLLV

SEQ ID NO: 9 - protein, 419 aa, Severe Acute Respiratory Syndrome Coronaviruses 2
  MISC_FEATURE (1)..(419): Nucleocapsid protein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 10 - DNA, 37 bp, Artificial Sequence
  Synthetic- TEE
catattgaagagacagagtgatatataaaactgctaa

SEQ ID NO: 11 - DNA, 42 bp, Artificial Sequence
  Synthetic- TEE
agaaccatattgaagagacagagtgatatataaaactgctaa

SEQ ID NO: 12 - DNA, 90 bp, Artificial Sequence
  Synthetic- TEE
agaaccatattgaagagacagagtgatatataaaactgctaactcaagcagcacaagaattaaatgaataccaagaaaatacttggccag

SEQ ID NO: 13 - protein, 9 aa, Artificial Sequence
  Synthetic- ebola glycoprotein epitope 6D8
VYKLDISEA

SEQ ID NO: 14 - DNA, 1047 bp, Artificial Sequence
  Synthetic- HBcAg-SARS-CoV-2-RBD
atggacattgacccttacaaagaatttggagctactgtggagcttctcagctttttgccttctgacttctttccttctgtcagggatctccttgacactgcctcagctctttatagggaagccttggagtctcctgagcattgctcacctcaccatactgcactcaggcaagccattctctgctggggagaattgatgactcttgctacctgggtgggtaacaatctagaggatccagcatccagagatcttgttgttaactatgttaatactaatgtgggtttgaagatcaggcaactcttgtggtttcatatatcttgccttacttttggaagagagactgtacttgaatatttggtctcttttggagtgtggattagaactcctccagcctatagaccaccaaatgcccctatcttgtcgactcttccagaaactactgttgtttctagaaatattactaatctttgccctttcggagaggtgtttaatgcaacaagatttgcatctgtttatgcttggaatagaaagagaatttctaactgtgttgctgattattcagttctttataattctgcttctttttctacattcaaatgttatggtgtgtcacctactaagttgaacgatctttgcttcactaatgtgtacgcagattctttcgttatcagaggagatgaggtgaggcagattgcaccaggtcaaactggtaaaatcgcagattataactacaaattgcctgacgacttcacaggttgtgtgattgcatggaactctaataacttggattcaaaagttggtggaaactacaactatttgtacagattgttcagaaaatcaaatcttaaaccatttgaaagagacatttctacagaaatataccaagctggttctacaccttgcaacggtgtggagggttttaattgttacttccctttgcagtcatacggattccaaccaacaaatggagttggatatcagccatacagagttgtggttttgtcttttgaattgttgcatgcaccagcaacagtgtgttaagagctc

SEQ ID NO: 15 - protein, 346 aa, Artificial Sequence
  Synthetic-HBcAg-SARS-CoV-2-RBD
MDIDPYKEFGATVELLSFLPSDFFPSVRDLLDTASALYREALESPEHCSPHHTALRQAILCWGELMTLATWVGNNLEDPASRDLVVNYVNTNVGLKIRQLLWFHISCLTFGRETVLEYLVSFGVWIRTPPAYRPPNAPILSTLPETTVVSRNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVC

SEQ ID NO: 16 - DNA, 2190 bp, Artificial Sequence
  Synthetic- Norwalk virus capsid protein
atgatgatggcttctaaggatgctacatcatctgtggatggagctagtggagctggtcaattggttccagaggttaatgcttctgaccctcttgctatggatcctgtagcaggttcttccacagcagttgctactgctggacaagttaatcctattgatccatggataattaacaactttgtgcaagccccccaaggtgaattcactatttccccaaacaacaccccaggtgatgttttgtttgatttgagtttgggtccccatcttaatcctttcttgctccatctctcacaaatgtataatggttgggttggtaacatgagagttaggattatgcttgctggtaatgcctttactgctggtaagataatagtttcttgcataccccctggttttggttcacataatcttactatagcacaagcaactctctttcctcatgtgattgctgatgttaggactcttgaccccattgaggtgcctttggaagatgttaggaatgttctctttcataacaacgatagaaatcaacaaaccatgaggcttgtgtgcatgctctacacccccttgaggactggtggtggtactggtgattcttttgtagttgcaggaagggttatgacttgcccaagtcctgattttaatttcttgtttttagtccctcctacagtggagcaaaaaaccaggcccttcacactcccaaatctcccattgagttctctctctaactcaagagcccctctcccaattagtagtatgggcatttccccagacaatgtccaaagtgtgcaattccaaaatggtaggtgtactcttgatggaagacttgttggcaccaccccagtaagcttgtcacatgttgccaagataagaggtacctccaatggcactgtgatcaaccttactgaattggatggcacaccctttcacccttttgagggccctgcccccattggatttccagatcttggtggttgtgattggcatatcaatatgacacaatttggccattctagccaaacccaatatgatgtcgacaccacccctgacacttttgtcccccatcttggttcaattcaagcaaatggcattggaagtggtaattatgttggtgttctttcttggatttcccccccatcacacccatctggctcccaagttgacctttggaagatccccaattatggatcaagtattactgaggcaacacatcttgccccttctgtatacccccctggttttggagaggtattggtctttttcatgtcaaaaatgccaggtcctggcgcttataatttgccatgtctcttaccacaagagtacatttcacatcttgctagcgagcaagcccctactgtaggtgaggctgccctgctccactatgttgaccctgatactggtaggaatcttggagaattcaaagcataccctgatggtttcctcacttgtgtccccaatggtgctagcagcggtccacaacaactgccaatcaatggtgtctttgtctttgtttcatgggtgtcaagattttatcaattaaagcctgtgggaactgcctctagcgcaagaggtaggcttggtcttaggaggtctagaaatattactaatctttgccctttcggagaggtgtttaatgcaacaagatttgcatctgtttatgcttggaatagaaagagaatttctaactgtgttgctgattattcagttctttataattctgcttctttttctacattcaaatgttatggtgtgtcacctactaagttgaacgatctttgcttcactaatgtgtacgcagattctttcgttatcagaggagatgaggtgaggcagattgcaccaggtcaaactggtaaaatcgcagattataactacaaattgcctgacgacttcacaggttgtgtgattgcatggaactctaataacttggattcaaaagttggtggaaactacaactatttgtacagattgttcagaaaatcaaatcttaaaccatttgaaagagacatttctacagaaatataccaagctggttctacaccttgcaacggtgtggagggttttaattgttacttccctttgcagtcatacggattccaaccaacaaatggagttggatatcagccatacagagttgtggttttgtcttttgaattgttgcatgcaccagcaacagtgtgttaagagctc

SEQ ID NO: 17 - protein, 727 aa, Artificial Sequence
  Synthetic- Norwalk virus capsid protein
MMMASKDATSSVDGASGAGQLVPEVNASDPLAMDPVAGSSTAVATAGQVNPIDPWIINNFVQAPQGEFTISPNNTPGDVLFDLSLGPHLNPFLLHLSQMYNGWVGNMRVRIMLAGNAFTAGKIIVSCIPPGFGSHNLTIAQATLFPHVIADVRTLDPIEVPLEDVRNVLFHNNDRNQQTMRLVCMLYTPLRTGGGTGDSFVVAGRVMTCPSPDFNFLFLVPPTVEQKTRPFTLPNLPLSSLSNSRAPLPISSMGISPDNVQSVQFQNGRCTLDGRLVGTTPVSLSHVAKIRGTSNGTVINLTELDGTPFHPFEGPAPIGFPDLGGCDWHINMTQFGHSSQTQYDVDTTPDTFVPHLGSIQANGIGSGNYVGVLSWISPPSHPSGSQVDLWKIPNYGSSITEATHLAPSVYPPGFGEVLVFFMSKMPGPGAYNLPCLLPQEYISHLASEQAPTVGEAALLHYVDPDTGRNLGEFKAYPDGFLTCVPNGASSGPQQLPINGVFVFVSWVSRFYQLKPVGTASSARGRLGLRRSRNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVC

SEQ ID NO: 18 - protein, 1273 aa, Artificial Sequence
  Synthetic- modified spike protein “Hexa Pro S”
  MISC_FEATURE (681)..(685): gsas mutation
  MISC_FEATURE (816)..(816): p mutation
  MISC_FEATURE (892)..(892): p mutation
  MISC_FEATURE (899)..(899): p mutation
  MISC_FEATURE (942)..(942): p mutation
  MISC_FEATURE (986)..(987): p mutation
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 19 - DNA, 3822 bp, Artificial Sequence
  Synthetic- modified spike protein “Hexa Pro S”
atgtttgtgtttcttgttttattaccattagtctctagtcagtgtgttaatttaacaaccagaactcaattaccccctgcatacactaattctttcacaagaggtgtttattaccctgacaaagttttcagatcctcagttttacattcaactcaggacttattcttacctttcttttccaatgttacttggttccatgctatacatgtctctgggaccaatggtactaagaggtttgataaccctgtcttaccatttaatgatggtgtttattttgcttccactgagaagtctaacataataagaggctggatttttggtactactttagattcgaagacccagtccttattaattgttaataacgctactaatgttgttattaaagtctgtgaatttcaattttgtaatgatccatttttaggtgtttattaccacaaaaacaacaaaagttggatggaaagtgagttcagagtttattctagtgcgaataattgcacttttgaatatgtctctcagccttttttaatggaccttgaaggaaaacagggtaatttcaaaaatttaagagaatttgtgtttaagaatattgatggttattttaaaatatattctaagcacacgcctattaatttagtgagagatctccctcagggtttttcggctttagaaccattggtagatttaccaataggtattaacatcactagatttcaaactttattagctttacatagaagttatttaactcctggtgattcttcttcaggttggacagctggtgctgcagcttattatgtgggttatttacaacctagaactttcttattaaaatataatgaaaatggaaccattacagatgctgtagactgtgcattagaccctttatcagaaacaaagtgtacgttaaaatccttcactgtagaaaaaggaatctatcaaacttctaactttagagtccaaccaacagaatctattgttagatttcctaatattacaaacttatgcccttttggtgaagtttttaacgccaccagatttgcatctgtttatgcttggaacagaaagagaatcagcaactgtgttgctgattattctgtcttatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatttagattctaaggttggtggtaattataattacttatatagattatttagaaagtctaatttaaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtattatcttttgaacttttacatgcaccagcaactgtttgtggacctaaaaagtctactaatttagttaaaaacaaatgtgtcaatttcaacttcaatggtttaacaggcacaggtgttttaactgagtctaacaaaaagtttctgcctttccaacaatttggcagagacattgctgacactactgatgctgtcagagatccacagacattagagattttagacattacaccatgttcttttggtggtgtcagtgttataacaccaggaacaaatacttctaaccaggttgctgttttatatcaggatgttaactgcacagaagtccctgttgctattcatgcagatcaattaactcctacttggagagtttattctacaggttctaatgtttttcaaacaagagcaggctgtttaataggggctgaacatgtcaacaactcatatgagtgtgacatacccattggtgcaggtatatgcgctagttatcagactcagactaattctcctgggagcgcaagtagtgtagctagtcaatccatcattgcctacactatgtcattaggtgcagaaaattcagttgcttactctaataactctattgccatacccacaaattttactattagtgttaccacagaaattttaccagtgtctatgaccaagacatcagtagattgtacaatgtacatttgtggtgattcaactgaatgcagcaatcttttgttgcaatatggcagtttctgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagagatcaccaattgaagatttacttttcaacaaagtgacattagcagatgctggcttcatcaaacaatatggtgattgcttaggtgatattgctgctagagacttaatttgtgcacaaaagtttaacggcttaactgttttaccacctttattaacagatgaaatgattgctcaatacacttctgcattattagcgggtacaatcacttctggttggacctttggtgcaggtccggcattacaaataccatttccgatgcaaatggcttatagatttaatggtattggagttacacagaatgttttatatgagaaccaaaaattaattgccaaccaatttaatagtgctattggcaaaattcaagactcattatcttccacaccaagtgcattaggaaaattacaagatgtggtcaaccaaaatgcacaagctttaaacacgttagttaaacaattaagctccaattttggtgcaatttcaagtgttttaaatgatatcttatcaagattagacccaccagaggctgaagtgcaaattgatagattaatcacaggcagattacaaagtttacagacatatgtgactcaacaattaattagagctgcagaaatcagagcttctgctaatttagctgctactaaaatgtcagagtgtgtattaggacaatcaaaaagagttgatttctgtggaaagggctatcatttaatgtccttccctcagtcagcacctcatggtgtagtcttcttacatgtgacttatgtccctgcacaagaaaagaacttcacaactgctcctgccatttgtcatgatggaaaagcacactttcctagagaaggtgtctttgtttcaaatggcacacactggtttgtaacacaaagaaatttctatgaaccacaaatcattactacagacaacacatttgtgtctggtaactgtgatgttgtaataggaattgtcaacaacacagtttatgatcctttacaacctgaattagactcattcaaggaggagttagataaatattttaagaatcatacatcaccagatgttgatttaggtgacatctctggcattaatgcttcagttgtaaacattcaaaaagaaattgacagattaaatgaggttgccaagaatttaaatgaatctttaatcgatttacaagaattaggaaagtatgagcagtatataaaatggccatggtacatttggttaggttttatagctggcttaattgccatagtaatggtgacaattatgttatgctgtatgaccagttgctgtagttgtttaaagggctgttgttcttgtggatcctgctgcaaatttgatgaagacgactctgagccagtgttaaaaggagtcaaattacattacacataa

SEQ ID NO: 20 - DNA, 90 bp, Artificial Sequence
  Synthetic- Primer for amplifying SARSCoV2-Hexa Pro spike
ggggacaacttttctatacaaagttgccaaaaattgaaattttattttttttttttggaatataaataatgtttgtgtttcttgttttat

SEQ ID NO: 21 - DNA, 54 bp, Artificial Sequence
  Synthetic- Primer for amplifying SARSCoV2-Hexa Pro spike
ggggacaactttattatacaaagttgttcattatgtgtaatgtaatttgactcc

SEQ ID NO: 22 - protein, 190 aa, Artificial Sequence
  Synthetic- SARS-CoV-2 receptor binding domain (RBD) G339D
  MISC_FEATURE (19)..(19): G339D mutation
QPTESIVRFPNITNLCPFDEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 23 - protein, 190 aa, Artificial Sequence
  Synthetic- SARS-CoV-2 receptor binding domain (RBD) S371L
  MISC_FEATURE (51)..(51): S371L mutation
QPTESIVRFPNITNLCPFGEVFNATRFASVYAWNRKRISNCVADYSVLYNLASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 24 - protein, 190 aa, Artificial Sequence
  Synthetic- SARS-CoV-2 receptor binding domain (RBD) S373P
  MISC_FEATURE (53)..(53): S373P mutation
QPTESIVRFPNITNLCPFGEVFNATRFASVYAWNRKRISNCVADYSVLYNSAPFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 25 - protein, 190 aa, Artificial Sequence
  Synthetic- SARS-CoV-2 receptor binding domain (RBD) S375F
  MISC_FEATURE (55)..(55): S375F mutation
QPTESIVRFPNITNLCPFGEVFNATRFASVYAWNRKRISNCVADYSVLYNSASFFTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 26 - protein, 190 aa, Artificial Sequence
  Synthetic- SARS-CoV-2 receptor binding domain (RBD) K417N
  MISC_FEATURE (97)..(97): K417N mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 27 - protein, 190 aa, Artificial Sequence
  Synthetic- SARS-CoV-2 receptor binding domain (RBD) K417T
  MISC_FEATURE (97)..(97): K417T mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 28 - protein, 190 aa, Artificial Sequence
  Synthetic- SARS-CoV-2 receptor binding domain (RBD) K417M
  MISC_FEATURE (97)..(97): K417M mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGMIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 29 - protein, 190 aa, Artificial Sequence
  Synthetic- SARS-CoV-2 receptor binding domain (RBD) N440K
  MISC_FEATURE (120)..(120): N440K  mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNKLDSKVGGNYNYLYRLFRKSNLKPFERDISTEIYQAGSTPCNGVEGFNCYFPLQSYGFQPTNGVGYQPYRV

SEQ ID NO: 30 - protein, 190 aa, Artificial Sequence
  Synthetic- SARS-CoV-2 receptor binding domain (RBD) G446S
  MISC_FEATURE (126)..(126): G446S mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVSGNYNYLYRLFRKSNLKPFERDISTEIYQAGSTPCNGVEGFNCYFPLQSYGFQPTNGVGYQPYRV

SEQ ID NO: 31 - protein, 190 aa, Artificial Sequence
  Synthetic- SARS-CoV-2 receptor binding domain (RBD) L452R
  MISC_FEATURE (132)..(132): L452R mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRV

SEQ ID NO: 32 - protein, 190 aa, Artificial Sequence
  Synthetic- SARS-CoV-2 receptor binding domain (RBD) S477N
  MISC_FEATURE (157)..(157): S477N mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGNTPCNGVEGFNCYFPLQSYGFQPTNGVGYQPYRV

SEQ ID NO: 33 - protein, 190 aa, Artificial Sequence
  Synthetic- SARS-CoV-2 receptor binding domain (RBD) T478K
  MISC_FEATURE (158)..(158): T478K mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSKPCNGVEGFNCYFPLQSYGFQPTNGVGYQPYRV

SEQ ID NO: 34 - protein, 190 aa, Artificial Sequence
  Synthetic- SARS-CoV-2 receptor binding domain (RBD) E484K
  MISC_FEATURE (164)..(164): E484K mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRV

SEQ ID NO: 35 - protein, 190 aa, Artificial Sequence
  Synthetic- SARS-CoV-2 receptor binding domain (RBD) E484A
  MISC_FEATURE (164)..(164): E484A mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVAGFNCYFPLQSYGFQPTNGVGYQPYRV

SEQ ID NO: 36 - protein, 190 aa, Artificial Sequence
  Synthetic- SARS-CoV-2 receptor binding domain (RBD) Q493K
  MISC_FEATURE (173)..(173): Q493K mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLKSYGFQPTNGVGYQPYRV

SEQ ID NO: 37 - protein, 190 aa, Artificial Sequence
  Synthetic- SARS-CoV-2 receptor binding domain (RBD) Q493R
  MISC_FEATURE (173)..(173): Q493R mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLRSYGFQPTNGVGYQPYRV

SEQ ID NO: 38 - protein, 190 aa, Artificial Sequence
  Synthetic- SARS-CoV-2 receptor binding domain (RBD) G496S
  MISC_FEATURE (176)..(176): Q496S mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYSFQPTNGVGYQPYRV

SEQ ID NO: 39 - protein, 190 aa, Artificial Sequence
  Synthetic- SARS-CoV-2 receptor binding domain (RBD) Q498R
  MISC_FEATURE (178)..(178): Q498R mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFRPTNGVGYQPYRV

SEQ ID NO: 40 - protein, 190 aa, Artificial Sequence
  Synthetic- SARS-CoV-2 receptor binding domain (RBD) N501Y
  MISC_FEATURE (181)..(181): N501Y mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRV

SEQ ID NO: 41 - protein, 190 aa, Artificial Sequence
  Synthetic- SARS-CoV-2 receptor binding domain (RBD) Y505H
  MISC_FEATURE (185)..(185): Y505H mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGHQPYRV